(19) United States
(12) Patent Application Publication
(10) Pub. No.: US 2006/0264856 A1
(43) Pub. Date: Nov. 23, 2006

(54) Method and liquid-filled apparatus for male masturbation

(76) Inventor: Ken E. Wong, Honolulu, HI (US)

Correspondence Address: KEN E. WONG, 5122 LIKINI ST. # 307, HONOLULU, HI, 96818, US

(21) Appl. No.: 11/132,753

(22) Filed: May 18, 2005

(51) Int. Cl. A61F 5/44

(52) U.S. Cl. 604/349

(57) ABSTRACT

A method and liquid-filled apparatus for male masturbation is disclosed. The method and apparatus incorporates an inner sheath (10) made from a pliable waterproof material that, when assembled within an outer containment vessel (12) by the application of a container sealing cap (14), forms a sealed chamber within the vessel (12) and outside the sheath (10) that can contain a warm liquid. The present method and apparatus creates an enhanced penile sensory experience by incorporating a fluid transmitting means by a predetermined number of holes (26) in the sheath (10) and a heat transferring means by the material from which the sheath (10) is constructed. By unscrewing the cap (14) from the vessel (12) and removing the sheath (10), an operator can conveniently clean the apparatus or reassemble the apparatus with other design variations of the sheath (10), vessel (12), and cap (12) made available to the operator to create a wide range of sensory experiences.

### BACKGROUND—FIELD OF INVENTION

This invention relates to a method and apparatus for device-assisted male masturbation, and more particularly to a method and apparatus for accommodating a male penis for the purpose of self gratification. Methods and apparatuses of this nature are of benefit to human males who, for whatever reasons, desire to experience the simulated sensations of sexual intercourse without the benefit of a female partner.

### BACKGROUND—DESCRIPTION OF PRIOR ART

Subsequent research has revealed that methods and apparatuses available to human males for self gratification have focussed on devices made of synthetic materials such as rubber in an attempt to simulate the actual experience of sexual intercourse. These methods which typically involve artificial replications of the cavity of the human female vagina are designed to accept and accommodate a male penis but do not fully simulate the range of penile sensations derived during actual human sexual intercourse.

U.S. Pat. No. 5,458,559 to Gauntlett shows an artificial vagina comprised of a sheath made from a flexible waterproof material which can be filled with warm water or other suitable fluid. However, this device lacks the versatility of allowing an operator the ability to experience a variety of sensations by interchanging variations of the aspect of the device that surrounds the penis during usage. In addition, the design of the apparatus is unnecessarily complex and also foregoes the opportunity to take advantage of a male tendency to be visually stimulated by the act of observing the penis during self gratification. In addition, no means is provided for the possibility of warm water or fluid movement from the device to the surface of the penis.

U.S. Pat. No. 5,836,865 to Ritchie shows a male sex aid. The aid is a tube made of three elongated inflated pillows joined to form a long open-ended artificial vagina. The pillows configure together to form a small triangular opening and orifice into which a penis can then be inserted. While Ritchie's invention can accommodate water, the triangular interior is not of the same shape as the interior of a woman's vagina. In addition, the device does not allow an operator to observe his penis during usage. Also, no means is provided for the possibility of warm water or fluid movement from the device to the surface of the penis.

U.S. Pat. No. 6,113,532 to Yap shows an ejaculation aid and sperm collection device. The aid and device is an airtight plastic shell with a cavity at the front end of the device for insertion of a penis. The device is inflated to a moderate pressure and the penis is then inserted into the cavity between the air chambers. The penis is stimulated by sliding the device on the penis. Manually squeezing of the outer walls during use helps to achieve the desired pressure. This device, however, does not provide for the penile sensations of warmth and wetness derived during copulation with a female vagina.

U.S. Pat. No. 6,415,937 to DeJong, U.S. Pat. No. 6,851,565 to Stephan, U.S. Pat. No. 6,089,389 to Sharon, and U.S. Pat. No. 5,024,341 to Dekerle which show prior art forms concerning bottle adapters and nipples for baby bottles were also examined.

While these inventions focus on unique adapter design variations for baby bottles, the intended use of these prior art forms is for securing an artificial nipple on the outside of a bottle for a baby to suckle on in order to draw liquid from the bottle. The prior art's use of a nipple is to draw liquid out of the bottle to allow a baby to suckle on a nipple positioned externally of the bottle. A baby cannot gain access to the nipple and, therefore, the liquid in the bottle if the nipple is turned inward inside the bottle. In the present invention, however, a new use of a sheath, which resembles a baby bottle nipple, requires that the sheath remain inside the bottle. This use of a sheath by the present invention has to the present moment been unobvious because the use of a nipple as an artificial vagina has not been considered up until now. Unlike a baby bottle nipple which is positioned outside of a bottle during usage, the inner sheath of the present invention is positioned inside a bottle during usage. Accidental discovery of this new use of a sheath by the present invention is unlikely because it would be illogical to present a hungry baby with a baby bottle with the nipple turned inside the bottle.

Furthermore, a hole or holes positioned on the sheath of the present invention provide further evidence of a new use method. It would be illogical for a baby bottle nipple with a hole to be used in the method 1 have proposed because the reverse flow of liquid from outside the nipple to the inside of the nipple (versus from inside the nipple to outside the nipple) is inconsistent with how a baby bottle is typically used. A baby bottle's nipple has a hole for the passage of liquid from inside the nipple to outside the nipple. In contrast, a hole or holes in the sheath of the present invention allows for the passage of liquid from outside the inner sheath into the inner sheath. The hole or holes used in this manner then allow liquid to lubricate the surface of a penis which has been inserted into the sheath.

The use of a container sealing cap, which resembles a baby bottle adapter, by the present invention has to the present moment also been unobvious because its use has not been considered up until now as a key component of an artificial vagina. The corresponding hole in a baby bottle adapter serves to secure a nipple protruding outwards through the hole for a baby to access during feeding. In the present invention, however, the hole in the cap serves a new use to allow for the insertion of a penis into the assembled device.

Thus, the patentability of the present method and apparatus can be argued based on the unexpected and unsuggested new uses of the disclosed member components to produce a method and liquid-filled apparatus for male masturbation.

### OBJECTS AND ADVANTAGES

Accordingly, several objects and advantages of the present invention are to provide penile sensations that realistically simulate actual sexual intercourse during the act of male self gratification. More specifically, the present invention provides the following objects and advantages:

(a) to provide a method and apparatus which simulates the temperature of the human body;

(b) to provide a method and apparatus which simulates the wetness and lubrication that occurs during sexual intercourse;

(c) to provide a method and apparatus which allows an operator to visually observe his penis during the act of self-gratification by use of the device;

(d) to provide a method and apparatus which is manually powered and does not require an alternative energy means to power locomotion;

(e) to provide a method and apparatus which utilizes hand and arm movements already familiar to human males who practice self-gratification with their hands and arms without the use of sexual devices or aids;

(f) to provide a method and apparatus which allows an operator to experience a variety of self-gratification penile sensations by interchangeably replacing component members with corresponding components with different design characteristics (examples: replacing an inner sheath with other sheaths of varying textures, shapes, materials, holes, no holes, etc.); and

(g) to provide a method and apparatus which utilizes component members which can easily be assembled, disassembled, cleaned, and reassembled.

Further objects and advantages of the present invention will become apparent from a consideration of the drawings and ensuing description.

### DRAWING FIGURES

FIG. 1 is a perspective view of the present invention comprising 3 members in a disassembled state: an inner sheath 10, an outer containing vessel 12, and a container sealing cap 14;

FIG. 2 is an exploded view of member components of the present invention and how they relate to one another during assembly and how the assembled configuration allows an operator to accomplish the present method of self gratification according to the invention fully assembled with a penis inserted;

FIG. 3 is a perspective view of possible design variations of member components including: an inner sheath 10, an outer containing vessel 12, and a container sealing cap 14; and

FIG. 4 is a perspective view of possible design variations of member components including: an inner sheath 10, an outer containing vessel 12, and a container sealing cap 14.

### REFERENCE NUMERALS IN DRAWINGS

10 inner sheath 12 outer containment vessel

14 container sealing cap 16 dome-shaped crown

18 intermediate segment 20 lower segment

22 annular base 24 annular flange

26 hole 28 threaded external neck ring

30 base 32 bottom end

34 top end 36 threaded inner receiving ring

38 cap hole 40 base hole

42 neck 44 open end

46 sheath protuberance 48 cap protuberance

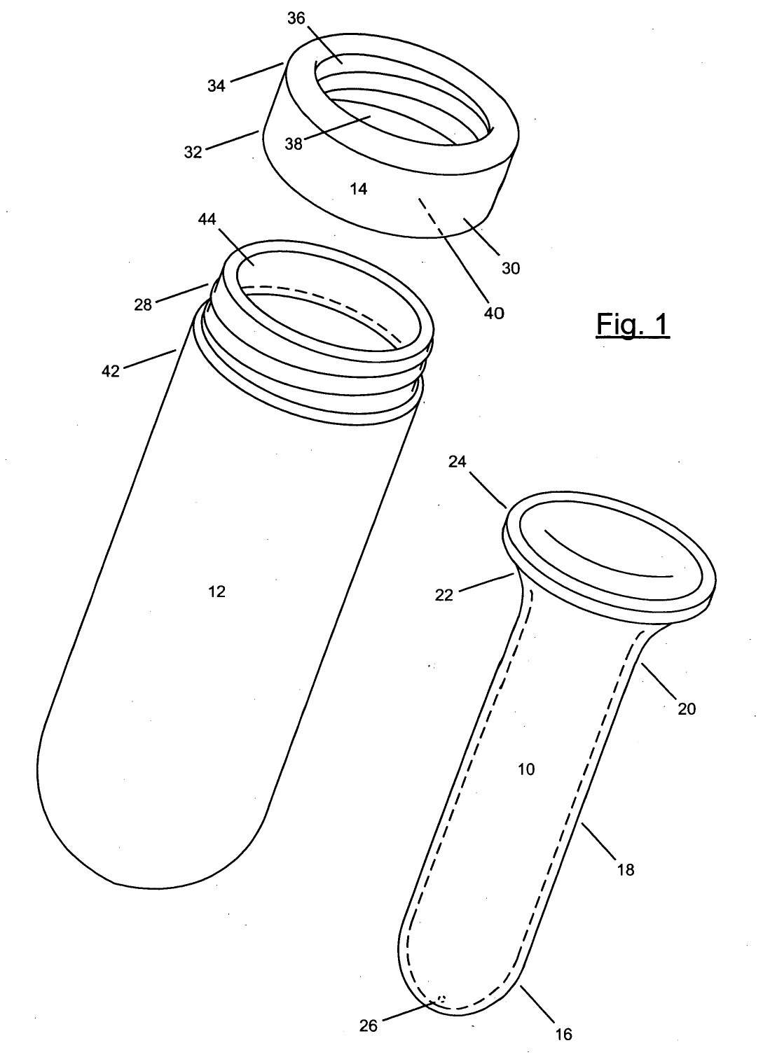

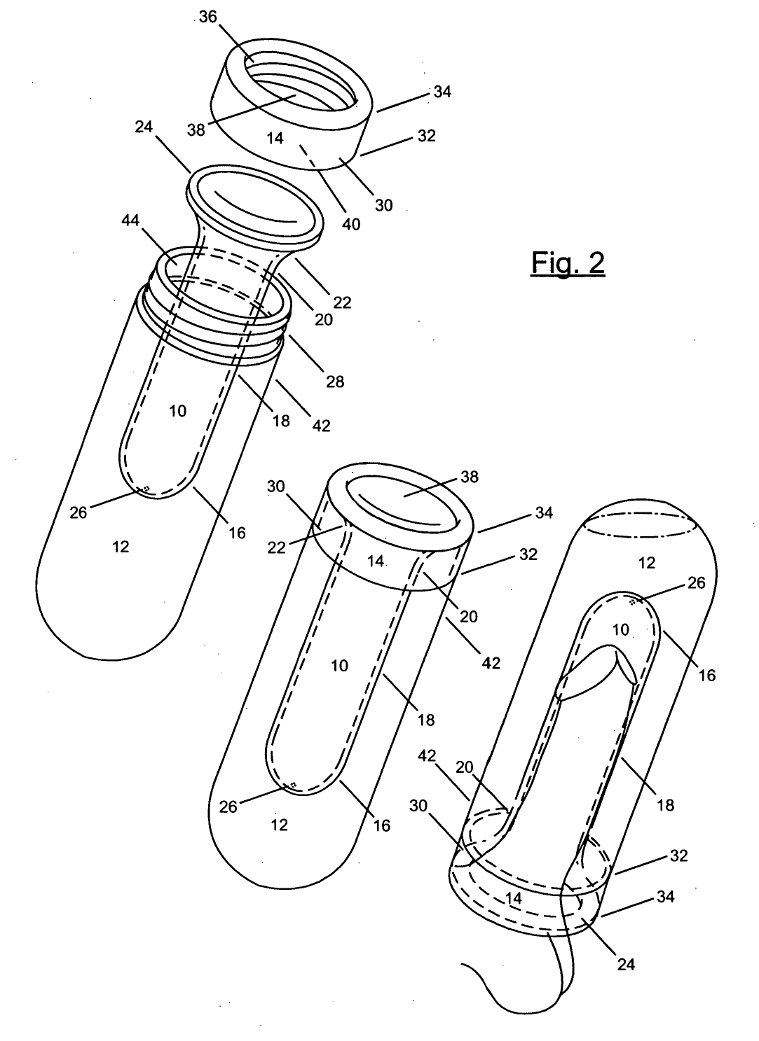

### DESCRIPTION—FIGS. 1 AND 2

FIG. 1 is a perspective view of the present invention comprised of 3 member components to which the present invention is applied: an inner sheath 10, an outer containment vessel 12, and a container sealing cap 14.

The inner sheath 10 is fabricated out of rubber, latex, or other pliable waterproof material and has a dome-shaped crown 16, an intermediate segment 18, and a lower segment 20 having an annular base 22 at a bottom edge of said lower segment 20, being the same diameter as dome-shaped crown 16 and intermediate segment 18. Under the annular base 22 at the bottom edge of the lower segment 20 is an annular flange 24 being of a diameter greater than said annular base 22 and equal to the inner diameter of a container sealing cap 14. A hole or holes 26 are incorporated into the inner sheath 10.

The container sealing cap 14 is fabricated out of a resilient material such as plastic, rubber, or latex and has a base 30 with a bottom end 32 and a top end 34. The bottom end 32 of the base 30 receives the outer containment vessel 12 and has a threaded inner receiving ring 36 to receive the threaded external neck ring 28 of the outer containment vessel 12. The top end 34 of the container sealing cap 14 includes a cap hole 38 for allowing the insertion of a penis into the inner sheath 10 when assembled within the outer containment vessel 12. The diameter of the cap hole 38 in the container sealing cap 14 is greater than the diameter of a human male penis and smaller than the diameter of the annular flange 24 of the inner sheath 10. A base hole 40 at the bottom end 32 of the container sealing cap 14 has a diameter greater than the diameter of the annular flange 24 of the inner sheath 10 and the open end 44 of the outer containment vessel 12.

The outer containment vessel 12 is fabricated out of a rigid or semi-pliable material to comprise a reservoir for receiving and containing a liquid such as water. The vessel 12 has a neck 42, and a free edge of which defines an open end 44. The reservoir having the open end 44 with a threaded external neck ring 28 for receiving the threaded inner receiving ring 36 of the container sealing cap 14 as shown in FIG. 2. For the purpose of the present invention, the outer containment vessel 12 may also be formed of a flexible or resilient material to create a deformable container with a sealed end. The use of a transparent materiala such as clear plastic or glass may be considered to enable an operator to observe his penis inside the present invention during usage.

Operation—FIG. 2

As shown in FIG. 2, to employ the method and apparatus for device-assisted male masturbation according to the present invention, an operator removes the container sealing cap 14 and inner sheath 10 from the outside containment vessel 12. According to FIG. 2, a container sealing cap 14 is first removed. According to FIG. 2, the rubber sheath 10 is then removed next. A liquid of a temperature preferred by the operator is then poured into the outer containment vessel 12. The inner sheath 10 is then replaced inside the outer containment vessel 12 containing the liquid. The container sealing cap 14 is then re-threaded over the open end of the outer containment vessel 12. When inverted with the capped end down, the device is perfectly sealed because of the presence of the container sealing cap 14 at the open end 44 of the outer containment vessel 12. The seal results from the proper positioning of the annular flange 24 of the inner sheath 10 between the container sealing cap 14 and top edge of a threaded external neck ring 28 of the outer containment vessel 12 as shown in FIG. 2.

The transference of liquid and heat into the inner sheath 10 is accompished as follows. The hole or holes 26 incorporated into the inner sheath 10 enable liquid surrounding the inner sheath 10 within the outer containment vessel 12 to enter the inner sheath 10. The decompression resulting from the movement of a penis in and out of the inner sheath 10 causes minute amounts of the liquid contained within the outer containment vessel 12 to be pumped into the inner sheath 10. Different sensations may be available to a male operator depending on variations concerning the holes (examples: size, number, position) when used in conjunction with various liquids (examples: water, oils, lotions, mixtures there of). Slits may also be incorporated and would serve a similar purpose to the holes 26. Heat from the liquid outside the inner sheath 10 is conducted into the inner sheath 10 by the material of the inner sheath 10.

Summary, Ramifications, and Scope

Accordingly, the reader will see that the method and apparatus of the liquid-filled design of this invention can be used to provide a human male with a method to achieve a self-gratification masturbation experience that makes possible the sensations of warmth, wetness, visual stimulation, all derived from a device which can be powered manually. Heat generated by the liquid in the outer containment vessel 12 warms the inner sheath 10 to simulate the internal temperature of a human body. Wetness is generated as the decompression resulting from a penis moving in and out of the inner sheath 10 causes minute amounts of the liquid to be drawn into the inner sheath 10, thus simulating bodily secretions typical of mucus linings of the vagina or mouth. The outer containment vessel 12, if constructed with a clear, transparent material allows an operator to maintain a view his penis within the outer containment vessel 12 and inner sheath 10. Typical hand and arm motions familiar to an operator with prior self-gratification experience without a sexual aid are appropriate to power the device. The outer containment vessel 12 in FIG. 1 may provide additional visual stimulation based on predetermined shapes.

Alternative design variations of the embodiments of FIGS. 1 and 2 are easily interchangeable to create a wide range of potential experiences. If an operator desires a variety of penile sensations, the operator may select an alternative design variation of the inner sheath 10. While the primary form of the inner sheath 10 is tubular and a predetermined number of holes 26 therethrough, basic structure may include variations in curvature, length, varying diameters along the entire length or certain sections of the sheath, and/or small slits to allow minute amounts of liquid from within the outer containment vessel 12 to drain into the inner sheath 10 during usage. Aferall, it is the overall object of the inner sheath 10 is to simulate penile sensations derived from contact with a human female vagina in terms of size, shape, pliability, and inner surface texture which a penis will come into contact with during sexual intercourse. Characteristics such as shapes or added materials may also be incorporated into the design of the container sealing cap 14. As the container sealing cap 14 comes into contact with an operator's pubic areas during use, design characteristics such as rounded shapes or added materials may be incorporated.

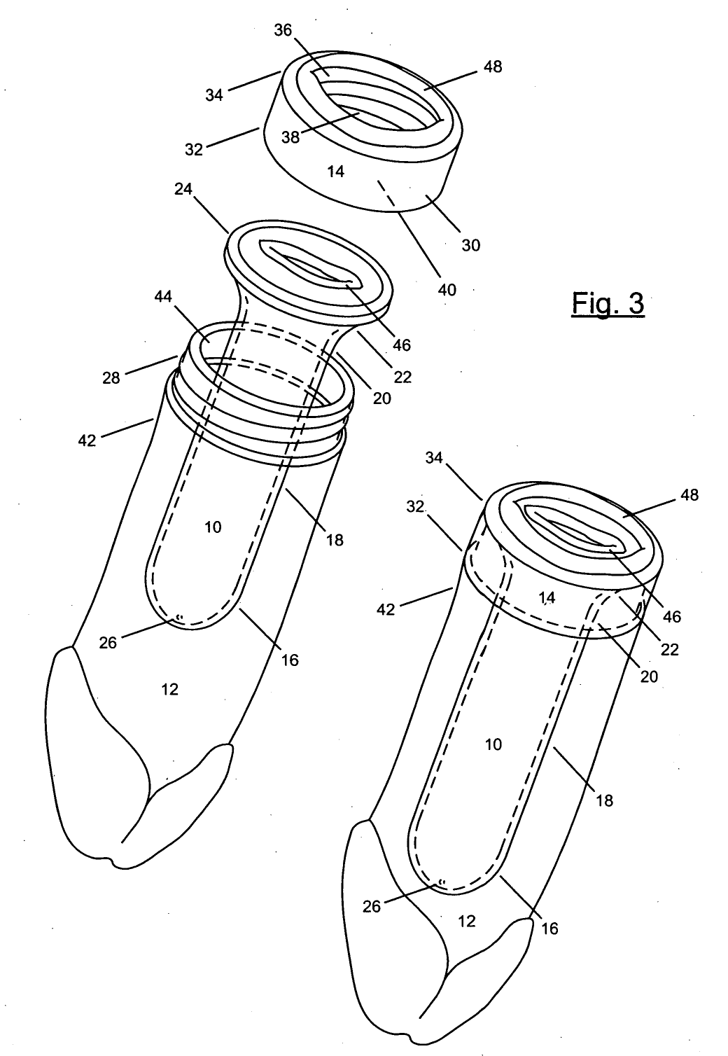

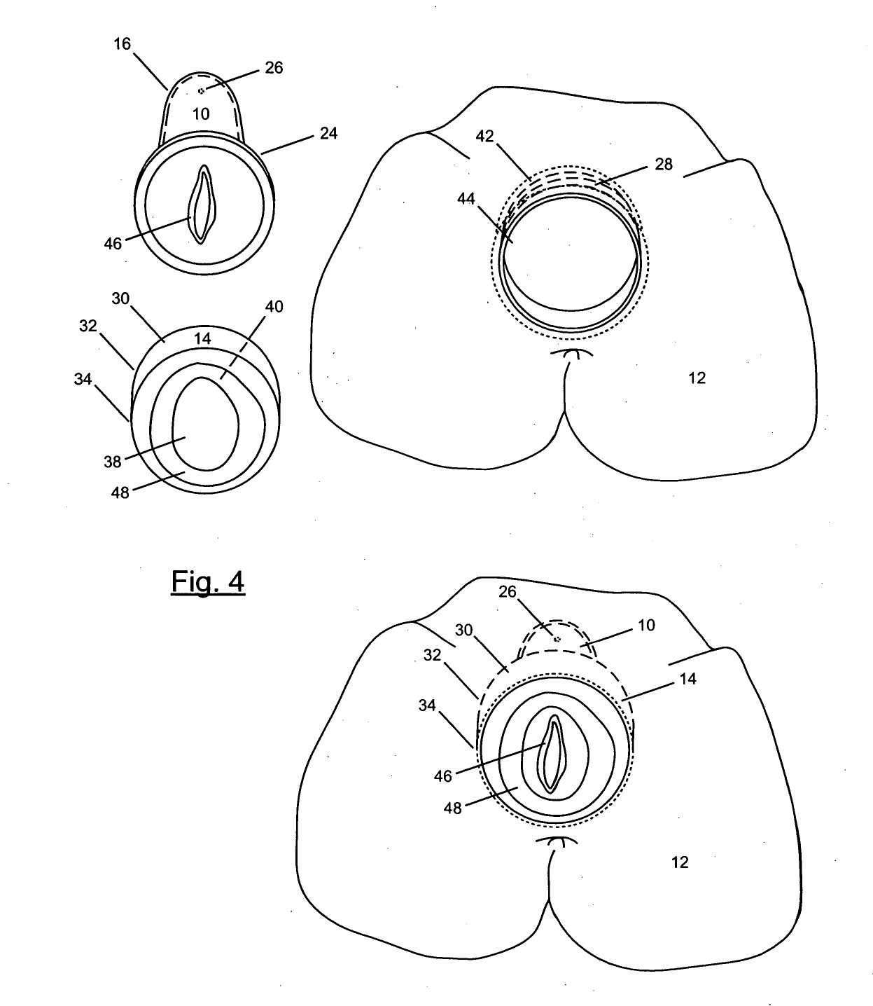

Thus, member components of the present invention can alternatively can be replaced by other members with different characteristics suited to a different type of self-gratification sensation or visual mode of application. As shown in FIGS. 3 and 4, these variations may include a sheath protuberance 46 that resembles a female anatomy's labia minora and a cap protuberance 48 that resembles a female anatomy's labia majora. As shown in FIG. 3, an outer containment vessel 12 may resemble a human male penis. An outer containment vessel 12 that resembles a human male penis additionally leads to the possible use of the present invention as an advanced form of condom. While a human male penis is inserted into the final assembly as shown in FIG. 3, the present invention itself may then be simultaneously inserted into a partner's body orifice. As shown in FIG. 4, an outer containment vessel 12 may resemble the pelvic area of a human female body. As with the member component designs shown in FIGS. 1 and 2, the same ease of assembly and disassembly also enables the member components of FIGS. 3 and 4 to be easily assembled, disassembled, cleaned and reassembled.

While my above description contains many specificities, these should not be construed as limitations on the scope of the invention, but rather as an exemplification of one preferred embodiment thereof. Many other variations are possible. Thus the reader will see that the method and apparatus utilizing the liquid-filled masturbation device of the present invention provides enhanced penile sensory stimulation for an operator during the act of self gratification by simulating those aspects of sexual intercourse which include warmth and wetness combined with an experience which is visually stimulating and which does not require the need for an external power source.

1-31. (canceled)

32. An apparatus for supplementing male self-gratification comprising a group of five physically independent structures: a tubular-shaped sheath with one open end having a flange and comprising a flexible waterproof material, a tubular-shaped containment vessel with one end and comprising a rigid waterproof material, a sealing cap open on two ends, an aqueous substance, and a sealed chamber.

33. The sheath of claim 32 further comprising a closed end having a predetermined number of apertures through which the aqueous substance of claim 32 can pass from outside said sheath to inside said sheath whereby said aqueous substance can facilitate lubrication within said sheath.

34. The flange at the open end of the sheath of claim 32 further in communication between the open end of the containment vessel of claim 32 and the distal open end of the cap of claim 32 whereby said sheath is sealed within said vessel.

35. The containment vessel of claim 32 further including male threads on the outside surface of the open end in communication with female threads on the inside surface of the sealing cap of claim 32.

36. The sealing cap of claim 32 further comprising a proximal and distal end each employing a hole of sufficient size for allowing the passage of an adult human male penis.

37. The aqueous substance of claim 32 further in communication between the outside surface of the sheath of claim 32 and the inside surface of the containment vessel of claim 32 and within the sealed chamber of claim 32.

38. The sealed chamber of claim 32 constructed by the communication of the flange at the open end of the sheath of claim 32, the open end of the containment vessel of claim 32, and the inside surface of the cap of claim 32.
